(19) United States
(12) Patent Application Publication
(10) Pub. No.: US 2012/0109306 A1
(43) Pub. Date: May 3, 2012

(54) SPINAL IMPLANT

(75) Inventors: Clinton R. Jacob, Memphis, TN (US); Mark C. Dace, Collierville, TN (US)

(73) Assignee: Warsaw Orthopedic, Inc., Warsaw, IN (US)

(21) Appl. No.: 12/914,514

(22) Filed: Oct. 28, 2010

(51) Int. Cl. A61F 2/44

(52) U.S. Cl. 623/17.16

(57) ABSTRACT

A spinal implant configured for implantation in an intervertebral disc space is disclosed. The implant comprises a section for engaging a vertebral body. The section comprises a bone-engaging surface configured to engage at least a portion of a vertical surface and/or an endplate surface of the vertebral body. The section further comprises a metal component and a polymer component that are bonded together, wherein each of the metal component and the polymer component comprises at least one structural element configured to promote fixation between the polymer component and the metal component.

The present disclosure is related to commonly owned U.S. Pat. No. 6,113,637 and U.S. Pat. No. 7,276,082, each of which is hereby incorporated herein by reference in its entirety.

### FIELD OF INVENTION

The present invention is directed to prosthetic implants.

### BACKGROUND

The present disclosure relates to spinal implants, and particularly, spinal implants for intervertebral space.

### SUMMARY OF THE INVENTION

A spinal implant configured for implantation in an intervertebral disc space is disclosed. The implant comprises a section for engaging a vertebral body. The section comprises a bone-engaging surface configured to engage at least a portion of a vertical surface and/or an endplate surface of the vertebral body. The section further comprises a metal component and a polymer component that are bonded together, wherein each of the metal component and the polymer component comprises at least one structural element configured to promote fixation between the polymer component and the metal component.

### BRIEF DESCRIPTION OF THE DRAWINGS

FIG. 1 is a schematic, cross-sectional view of two adjacent vertebral bodies;

FIG. 2 is an isometric view of a spinal implant;

FIG. 3 is an exploded, isometric view of the spinal implant of FIG. 2;

FIG. 4 is a cross-sectional view of the spinal implant of FIG. 2;

FIG. 5 is a cross-sectional view of the exploded, isometric view of the spinal implant of FIG. 3;

FIG. 6 is a schematic, cut-away, cross-sectional view of the spinal implant of FIG. 4;

FIG. 7 is a schematic, cut-away, cross-sectional view of another spinal implant;

FIG. 8 is a schematic, cut-away, cross-sectional view of another spinal implant; and

FIG. 9 is a schematic, cut-away, cross-sectional view of another spinal implant.

### DETAILED DESCRIPTION

For the purposes of promoting an understanding of the principles of the invention, reference will now be made to the embodiments, or examples, illustrated in the drawings and specific language will be used to describe the same. It will nevertheless be understood that no limitation of the scope of the invention is thereby intended. Any alterations and further modifications in the described embodiments, and any further applications of the principles of the invention as described herein are contemplated as would normally occur to one skilled in the art to which the invention relates.

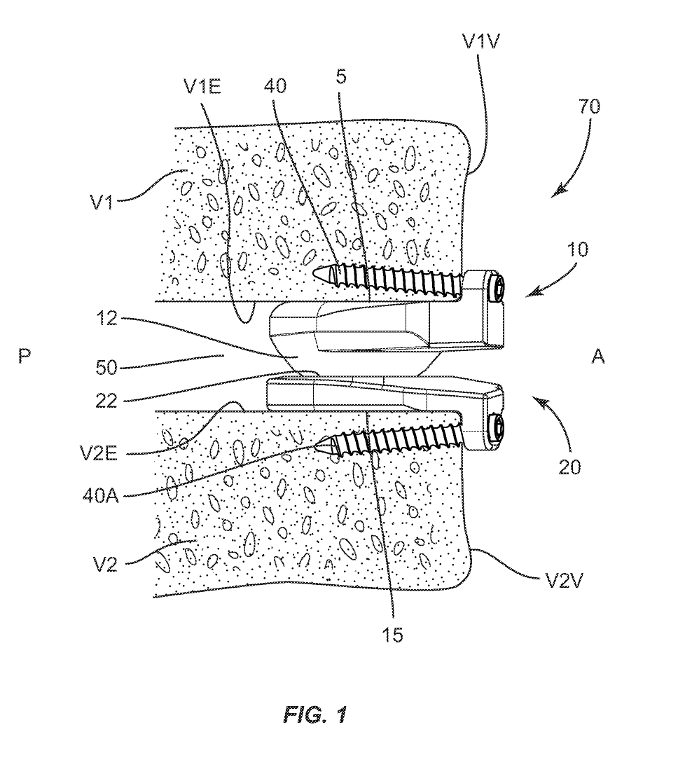

FIG. 1 shows a schematic, cross-sectional view of two adjacent vertebral bodies V1 and V2 with an intervertebral disc space 50 situated in its natural location between the two vertebral bodies V1 and V2. As shown in FIG. 1, vertebral body V1 represents a superior vertebral body and V2 represents an inferior vertebral body. Reference marker A represents an anterior side of the vertebral bodies V1 and V2, whereas reference marker P represents a posterior side of the vertebral bodies V1 and V2.

Also, FIG. 1 shows a spinal implant 70 situated in the disc space 50. As shown, spinal implant 70 comprises a first section 10 and a second section 20. The first section 10 is configured for engaging vertebral body V1, which is a superior vertebral body V1. The second section 20 is configured for engaging vertebral body V2, which is an inferior vertebral body V2. Specifically, as shown in FIG. 1, the first section 10 comprises a bone-engaging surface 5 for engaging an endplate surface V1E and a vertical surface V1V of superior vertebral body V1. Similarly, as shown in FIG. 1, the second section 20 comprises a bone-engaging surface 15 for engaging an endplate surface V2E and a vertical surface V2V of inferior vertebral body V2.

In addition, as shown in FIG. 1, there is a first fastener 40 for promoting fixation between the first component 10 and the superior vertebral body V1. Similarly, as shown, there is a second fastener 40A for promoting fixation between the second component 20 and the inferior vertebral body V2.

Further, as shown in FIG. 1, the first section 10 comprises a non-bone-engaging surface 12 and the second section comprises a non-bone-engaging surface 22. Specifically, as shown in FIG. 1, the non-bone-engaging surface 12 comprises a portion of the surface of a ball 12 or a spherically-shaped surface 12 and the non-bone-engaging surface 22 comprises a trough-shaped surface 22. Each of the ball surface 12 and the trough surface 22 are configured for articulation with one another so that motion is preserved between vertebral bodies V1 and V2.

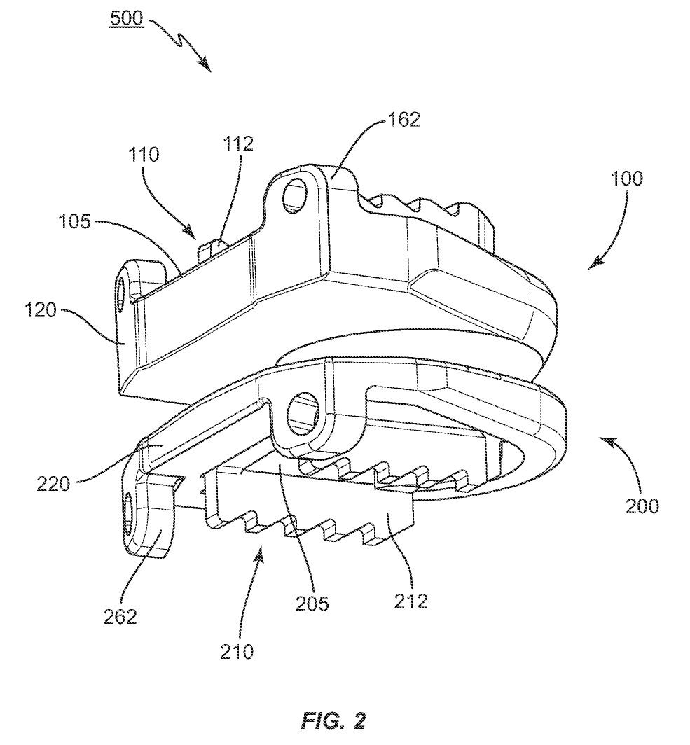

FIG. 2 shows an isometric view of a spinal implant 500. Spinal implant 500 is configured for implantation in an intervertebral disc space 50 and for preserving motion therein. Spinal implant 500 comprises a first section 100 for engaging a superior vertebral body V1 and a second section 200 for engaging an inferior vertebral body V2. As shown in FIG. 2, the first section 100 comprises a bone-engaging surface 105 configured to engage at least a portion of the vertical surface V1V and/or the endplate surface V1E of superior vertebral body V1. Similarly, the second section 200 comprises a bone-engaging surface 205 configured to engage at least a portion of the vertical surface V2V and/or the endplate surface V2E of inferior vertebral body V2. In addition, the first section 100 comprises an articulating surface and the second section 200 comprises an articulating surface, each articulating surface configured to engage the articulating surface of the other section.

As shown in FIG. 2, the first section 100 and the second section 200 each comprise a metal component and a polymer component that are bonded together. The first section 100 comprises a polymer component 120 and a metal component 110, whereas the second section 200 comprises a polymer component 220 and a metal component 210. As shown in FIG. 2, each metal component 110 and 210 comprise anchors 112 and 212 that extend from the bone engaging surface 105 and 205, respectively, and are configured for promoting fixation between the bone engaging surface 105 and 205 and the endplate surface V1E and V2E of vertebral bodies V1 and V2, respectively. Similarly, the metal components 110 and 210 of spinal implant 500 are configured for contact with the vertebral body.

Further, as shown, the first component 100 and the second component 200 have lugs 162 and 262, respectively, for use during insertion of the spinal implant 500 into the disc space 50 by, for example, providing locations for an insertion instrument to hold. In addition, lugs 162 and 262 may be helpful in preventing unwanted movement of the sections 100 and 200, respectively. That is, for example, if the spinal implant is inserted in an anterior approach, lugs 162 and 262 may prevent unwanted movement in the posterior direction.

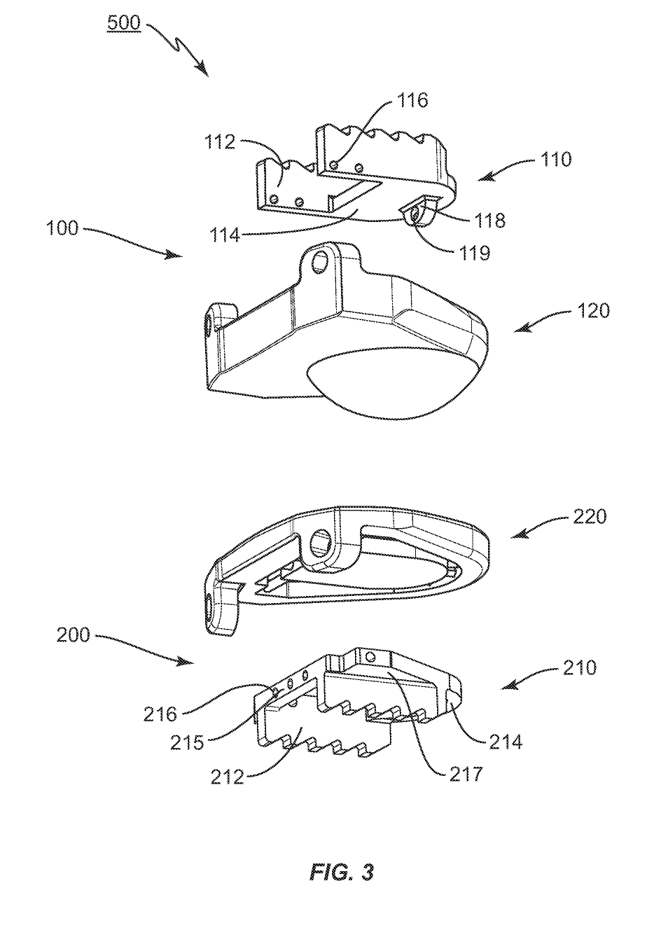

FIG. 3 shows an exploded, isometric view of the spinal implant 500 of FIG. 2. FIG. 3 shows the metal component 110 and the polymer component 120 of the first section 100, and shows the metal component 210 and the polymer component 220 of the second section 200. As shown in FIG. 3, the metal component 110 and the polymer component 120 of the first section 100 comprise at least one structural element configured to promote fixation between the polymer component 120 and the metal component 110. Similarly, the metal component 210 and the polymer component 220 of the second section 200 comprise at least one structural element configured to promote fixation between the polymer component 220 and the metal component 210.

Specifically, as shown in FIG. 3, the metal component 110 comprises anchors 112, a bridging member 114 connecting the two anchors 112, a vertical flange 118 extending from the bridging member 114, a plurality of holes 116 in the anchors 112 and a hole 119 in the vertical flange 118. Similarly, the metal component 210 comprises anchors 212, an anterior bridging member 215 connecting the two anchors 212 on an anterior side of the implant 500, a posterior bridging member 214 connecting the two anchors 212 on a posterior side of the implant 500, two lateral flanges 217 extending from the anchors 212 and a plurality of holes 216 in the anterior bridging member 215. All of these elements may serve as structural elements—configured to promote fixation between the polymer components 120 and 220 and the metal components 110 and 220, respectively. That is, during manufacturing, first the metal components 110 and 210 are formed, for example, by machining. Then, the metal components 110 and 210 are placed in separate molds having cavities or holes for the introduction of polymer material. The polymer material is inserted or poured, as the polymer is in a relatively hot, liquid state at this point in manufacturing process. The liquid polymer fills in all of the spaces surrounding the metal components 110 and 210, including, for example, in and around holes 116, 119 and 216, and adjacent and around flanges 118 and 217, bridging members 114, 214 and 215 and the bases of anchors 112 and 212, thereby forming complementary structural elements in the polymer components 120 and 220, respectively. Thus, after allowing time to cool and dry, the respective sections 100 and 200 take on their intended, final shape, as shown in FIG. 2. In the intended, final shape, the polymer components 120 and 220 may reside in and surround holes 116, 119 and 216 of metal components 110 and 210, and at least partially surround or completely surround flanges 118 and 217, bridging members 114, 214 and 215 and the bases of anchors 112 and 212.

Note that structural elements may take on a variety of types, shapes and sizes. For example, the holes 116, 119 and 216 of metal components 110 and 210 need not be circular, but may have other shapes, for example, the shape of a rectangle, square or diamond, to name a few. One limitation may be the overall shape of the spinal implant to incorporate structural elements. That is, the intended shape and size of the first and second sections 100 and 200 of spinal implant 500 may dictate the type, shapes and sizes of the structural elements used to promote fixation between the polymer and metal components.

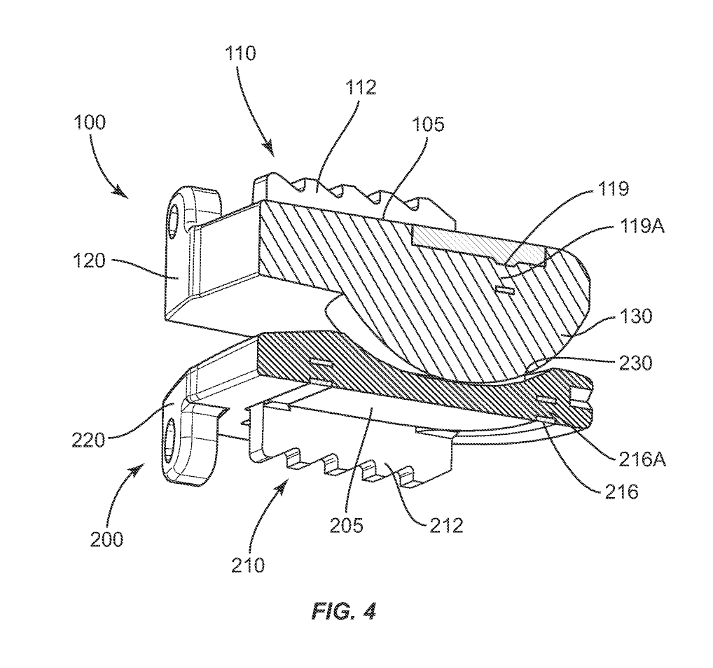

FIG. 4 shows a cross-sectional view of the spinal implant 500 of FIG. 2 taken in the A-P direction. As shown in FIG. 4, the metal component 110 comprises the structural element 119, namely hole 119, and the polymer component 120 comprises respective complementary structural element 119A. Further, shown in FIG. 4, the metal component 210 comprises the structural element 216, namely holes 216, and the polymer component 120 comprises respective complementary structural elements 216A.

As shown in FIG. 4, the first section 100 comprises a bone-engaging surface 105 configured to engage at least a portion of the vertical surface V1V and/or the endplate surface V1E of superior vertebral body V1. Similarly, the second section 200 comprises a bone-engaging surface 205 configured to engage at least a portion of the vertical surface V2V and/or the endplate surface V2E of inferior vertebral body V2. In addition, FIG. 4 shows the articulating surface 130 of the first section 100 and the articulating surface 230 of the second section 200, each articulating surface configured to engage the articulating surface of the other section. In this way, spinal implant 500 is similar to spinal implant 70 of FIG. 1 in that they both have ball and trough articulating surfaces. One difference between the spinal implants 70 and 500, however, is that spinal implant 500 does not utilize fasteners 40 and 40A. In lieu of the fasteners 40 and 40A, spinal implant 500 utilizes, for example, anchors 112 and 212. Another difference between the two spinal implants is that spinal implant 70 does not have lugs such as lugs 162 and 262 of spinal implant 500.

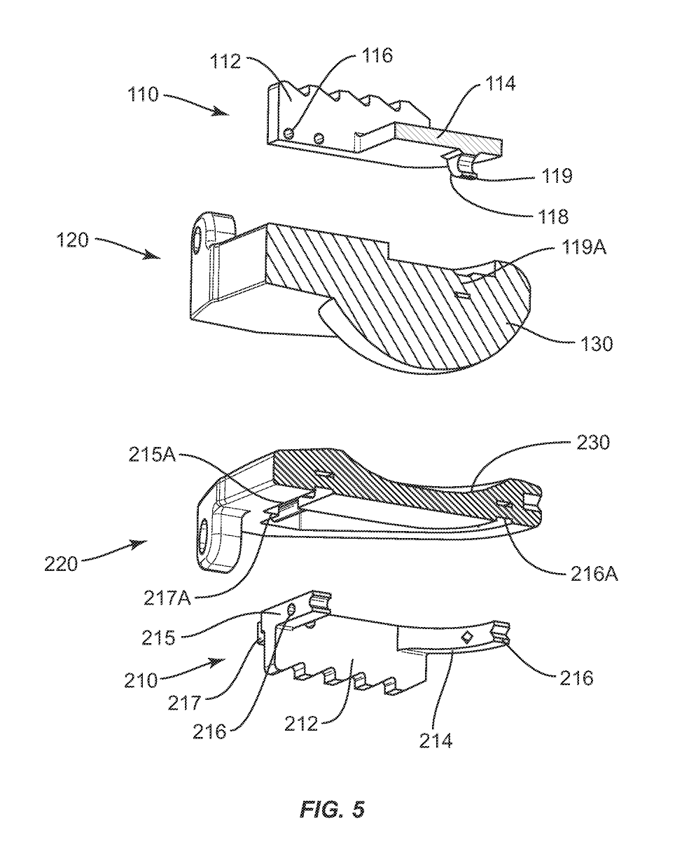

FIG. 5 shows a cross-sectional view of the exploded, isometric view of the spinal implant 500 of FIG. 3 taken in the A-P direction. As shown in FIG. 5, the metal component 110 comprises, for example, the structural element 119, namely hole 119, and the polymer component 120 comprises respective complementary structural element 119A. Further, as shown in FIG. 5, the metal component 210 comprises, for example, the structural element 216, namely holes 216, and the polymer component 120 comprises respective complementary structural elements 216A. In addition, as shown, the metal component 210 comprises, for example, the structural elements 215 and 217, namely bridging member 215 and flange 217, and the polymer component 120 comprises respective complementary structural elements 215A and 217A, respectively.

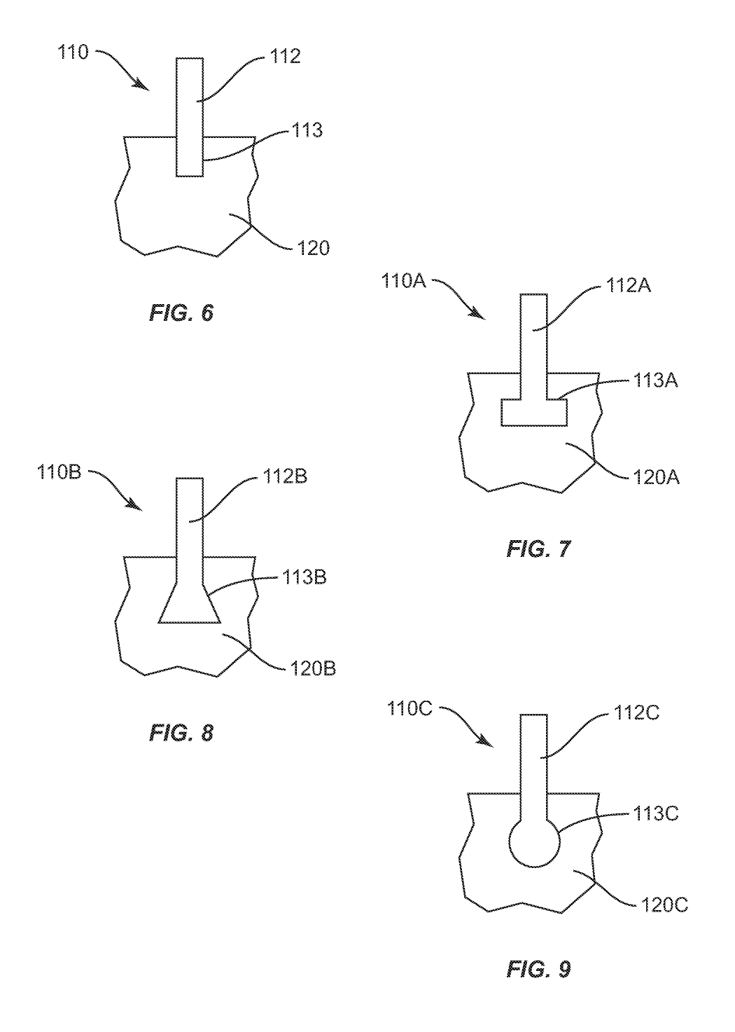

FIG. 6 shows a schematic, cut-away, cross-sectional view of the spinal implant 500 of FIG. 4 taken in a lateral direction. Specifically, FIG. 6 shows anchor 112 of the metal component 110 embedded in the polymer component 120. More specifically, as shown in FIG. 6, anchor 112 comprises a base 113 and it is the base 113 that is embedded in the polymer component 120. As shown in FIG. 6, anchor 112 comprises a structural element, namely base 113, which has a substantially rectangular cross section. As shown in FIG. 6, base 113 and polymer component 120 has a shape similar to a tongue and groove joint.

FIG. 7 shows a schematic, cut-away, cross-sectional view of another spinal implant taken in a lateral direction. Specifically, FIG. 7 shows an alternate embodiment of anchor 112, namely anchor 112A of metal component 110A. As shown in FIG. 7, anchor 112A comprises a base 113A that is embedded in the polymer component 120A. As shown in FIG. 7, anchor 112A comprises a structural element, namely base 113A, which has a substantially T-shaped cross section.

FIG. 8 shows a schematic, cut-away, cross-sectional view of another spinal implant taken in a lateral direction. Specifically, FIG. 8 shows an alternate embodiment of anchor 112, namely anchor 112B of metal component 110B. As shown in FIG. 8, anchor 112B comprises a base 113B that is embedded in the polymer component 120B. As shown in FIG. 8, anchor 112B comprises a structural element, namely base 113B, which has a substantially angled or substantially triangular cross section. As shown in FIG. 8, base 113B and polymer component 120B has a shape similar to a dovetail joint.

FIG. 9 shows a schematic, cut-away, cross-sectional view of another spinal implant taken in a lateral direction. Specifically, FIG. 9 shows an alternate embodiment of anchor 112, namely anchor 112C of metal component 110C. As shown in FIG. 9, anchor 112C comprises a base 113C that is embedded in the polymer component 120C. As shown in FIG. 9, anchor 112C comprises a structural element, namely base 113C, which has a substantially rounded or substantially circular cross section.

The term “substantially” as used herein may be applied to modify any quantitative representation which could permissibly vary without resulting in a change in the basic function to which it is related. For example, the base 113C of the anchor 112C may be considered substantially rounded or substantially circular if when the base 113C of anchor 112C is in its fully-embedded position in polymer component 120C, the rounded portion or circular portion of the base 113C helps promote fixation between the polymer component 120C and the metal component 110C.

Among other benefits, one benefit of being able to use some metal material with polymer materials in an implant (or using some metal material with a polymer-based implant) is that while a polymer material may be radiolucent, a typical metal may not be radiolucent. When examined with Magnetic Resonance Imaging (“MRI”), a typical metal may cause some distortion, whereas a typical polymer may cause less or no distortion. In addition, generally, metal surfaces provide better surfaces for bone in-growth, or bone to grow. That is, typically, bone may grow up to and onto a surface made of a metal such as Titanium, but not as much with a surface made of a polymer such as PEEK. Further, compared to surface coatings of metal material, a portion of an implant made of metal may provide greater structural stability and load bearing properties than a mere surface coating of metal.

The spinal implant 500 described herein may be made of a variety of biocompatible materials and combinations of materials. Some polymer materials used for the polymer components 120 and/or 220 may include, but not be limited to, any one or combination of polyetheretherketone (“PEEK”), polyetherketoneketone (“PEKK”), ultra high molecular weight polyethylene (“UHMWPE”), polyethylene, polyester, polyvinyl, polyvinyl alcohol, polyacrylonitrile, polyamide, polytetrafluoroethylene, poly-paraphenylene, polyurethane and terephthalamide.

Some materials used for the metal 110 and/or 210 may include, but not be limited to, any one or combination of Titanium or Titanium alloys such as “Ti6Al4V,” cobalt chrome alloy (“CoCr”), stainless steel and other suitable metals or metal alloys.

All adjustments and alternatives described above are intended to be included within the scope of the invention, as defined exclusively in the following claims. Those skilled in the art also should realize that such modifications and equivalent constructions or methods do not depart from the spirit and scope of the present disclosure, and that they may make various changes, substitutions, and alterations herein without departing from the spirit and scope of the present disclosure. For example, although the spinal implants described herein are with reference to spinal implants for preserving motion, note that a spinal implant with characteristics disclosed herein also may be used for spinal implants that promote fusion. Further, it is understood that all spatial references, such as “superior,” “inferior,” “anterior,” “posterior,” “lateral,” and “vertical” are for illustrative purposes only and can be varied within the scope of the disclosure.

1. A spinal implant configured for implantation in an intervertebral disc space, the implant comprising a section for engaging a vertebral body, the vertebral body comprising a vertical surface and an endplate surface, the section comprising:
a bone-engaging surface configured to engage at least a portion of the vertical surface and/or the endplate surface,
wherein the section comprises a metal component and a polymer component that are bonded together, and
wherein each of the metal component and the polymer component comprises at least one structural element configured to promote fixation between the polymer component and the metal component.

2. The spinal implant of claim 1, wherein the metal component is configured for contact with the vertebral body.

3. The spinal implant of claim 1, wherein at least a portion of the at least one structural element of the metal component has a substantially rectangular cross section.

4. The spinal implant of claim 1, wherein at least a portion of the at least one structural element of the metal component has a substantially triangular cross section.

5. The spinal implant of claim 1, wherein at least a portion of the at least one structural element of the metal component has a substantially rounded cross section.

6. The spinal implant of claim 1, wherein at least a portion of the at least one structural element of the metal component has a substantially circular cross section.

7. The spinal implant of claim 1, wherein the at least one structural element of the metal component is a hole.

8. The spinal implant of claim 1, wherein the vertebral body is a superior vertebral body and wherein the section is a first section for engaging the superior vertebral body, wherein the implant further comprises:
a second section for engaging an inferior vertebral body, the inferior vertebral body comprising a vertical surface and an endplate surface, the second section comprising:
a bone-engaging surface configured to engage at least a portion of the vertical surface and/or the endplate surface of the inferior vertebral body,
wherein the second section comprises a second metal component and a second polymer component that are bonded together, and
wherein each of the metal component and the polymer component comprises at least one structural element configured to promote fixation between the polymer component and the metal component.

9. The spinal implant of claim 1, wherein the spinal implant promotes fusion.

10. A spinal implant configured for implantation in a intervertebral disc space and preserving motion therein, the implant comprising a first section for engaging a superior vertebral body and a second section for engaging an inferior vertebral body, each of the superior and inferior vertebral bodies comprising a vertical surface and an endplate surface, each section comprising:
a bone-engaging surface configured to engage at least a portion of the vertical surface and/or the endplate surface; and
an articulating surface configured to engage the articulating surface of the other section;
wherein each section comprises a metal component and a polymer component that are bonded together, respectively, and
wherein each of the metal component and the polymer component of at least one of the first section and second section comprises at least one structural element configured to promote fixation between the polymer component and the metal component.

11. The spinal implant of claim 10, wherein the metal component of the first section is configured for contact with the superior vertebral body, and wherein the metal component of the second section is configured for contact with the inferior vertebral body.

12. The spinal implant of claim 10, wherein the at least one structural element of the polymer component complements the at least one structural element of the metal component.

13. The spinal implant of claim 10, wherein at least a portion of the at least one structural element of the metal component is a hole.

14. The spinal implant of claim 13, wherein at least a portion of the at least one structural element of the polymer component resides in and at least partially surrounds the hole of the metal component.

15. The spinal implant of claim 12, wherein the at least one structural element of the metal component has a substantially rectangular cross section and the at least one structural element of the polymer component at least partially surrounds the at least one structural element of the metal component.

16. The spinal implant of claim 11, wherein the at least one structural element of the metal component has a substantially triangular cross section and the at least one structural element of the polymer component at least partially surrounds the at least one structural element of the metal component.

17. The spinal implant of claim 11, wherein the at least one structural element of the metal component has a substantially rounded cross section and the at least one structural element of the polymer component at least partially surrounds the at least one structural element of the metal component.

18. The spinal implant of claim 11, wherein the at least one structural element of the metal component has a substantially circular cross section and the at least one structural element of the polymer component at least partially surrounds the at least one structural element of the metal component.

19. A spinal implant configured for implantation in a intervertebral disc space and preserving motion therein, the implant comprising a first section for engaging a superior vertebral body and a second section for engaging an inferior vertebral body, each of the superior and inferior vertebral bodies comprising a vertical surface and an endplate surface, each section comprising:
a bone-engaging surface configured to engage at least a portion of the vertical surface and/or the endplate surface; and
an articulating surface configured to engage the articulating surface of the other section;
wherein each section comprises a metal component and a polymer component that are bonded together, respectively, wherein the metal component comprises anchors that extend from the bone engaging surface and configured for promoting fixation between the bone engaging surface and the endplate of the respective vertebral body; and
wherein each of the metal component and the polymer component of at least one of the first section and second section comprises at least one structural element configured to promote fixation between the polymer component and the metal component.

20. The spinal implant of claim 19, wherein the at least one structural element of the polymer component complements the at least one structural element of the metal component.
